(19) United States
(12) Patent Application Publication
(10) Pub. No.: US 2013/0184179 A1
(43) Pub. Date: Jul. 18, 2013

(54) SMALL MOLECULE ARRAYS AND METHODS FOR MAKING AND USING THEM

(71) Applicants: Dayu Teng, San Diego, CA (US); Shu Chien, La Jolla, CA (US); Santosh Kesari, San Diego, CA (US); Pengfei Jiang, San Diego, CA (US)

(72) Inventors: Dayu Teng, San Diego, CA (US); Shu Chien, La Jolla, CA (US); Santosh Kesari, San Diego, CA (US); Pengfei Jiang, San Diego, CA (US)

(73) Assignee: THE REGENTS OF THE UNIVERSITY OF CALIFORNIA, Oakland, CA (US)

(21) Appl. No.: 13/817,959

(22) PCT Filed: Aug. 19, 2011
(86) PCT No.: PCT/US11/48395

§ 371 Date: Apr. 3, 2013

Related U.S. Application Data
(60) Provisional application No. 61/375,373, filed on Aug. 20, 2010.

(51) Int. Cl. G01N 33/50

(52) U.S. Cl. CPC G01N 33/5008
USPC 506/10; 506/15; 506/30

(57) ABSTRACT

In alternative embodiments, the invention provides products of manufacture, such as arrays or microarrays, comprising cells and compounds such as small molecules or drugs for e.g., drug screening or toxicity testing.

### RELATED APPLICATIONS

This application is a national phase application claiming benefit of priority under 35 U.S.C. §371 to Patent Convention Treaty (PCT) International Application Serial No: PCT/US2011/048395, filed Aug. 19, 2011, published as WO/2012/024574, which claims benefit of priority to U.S. Provisional Patent Application Ser. No. (“USSN”) 61/375,373, filed Aug. 20, 2010. The aforementioned applications are expressly incorporated herein by reference in their entirety and for all purposes.

### TECHNICAL FIELD

This invention generally relates to drug development and biochemistry. In alternative embodiments, the invention provides products of manufacture, such as arrays or microarrays, comprising cells and compounds such as small molecules or drugs for e.g., drug screening or toxicity testing.

### BACKGROUND

Conventional high throughput small molecule screening is conducted using a liquid handling robot in multi-well format. Typically, the cells are cultured in multi-well plates, e.g., 96 to 3456 wells; and small molecules are added into each well. The unit culture area in each well (e.g. for a 3456 well-plate) is about 3 mm² for each compound. A costly liquid-handling robot is required for large scale screening. Each test requires 1 μl of the compound.

A conventional microarray drug screening immobilizes drugs on the glass slide in poly(dl-lactide-coglycolide) (PLGA), then seeds cells covering the whole slide. The effective drug will interfere with the cell survival and proliferation, resulting in a cell-less spot. However, it requires a larger number of cells to cover the whole slide. Because there is no barrier between each spot, the cells are free to migrate from one spot to another. The cells affected by different spot may interact and lead to less independent experiments. The lack of barrier also causes a less defined reading area, making quantification more difficult and less accurate.

### SUMMARY

In alternative embodiments, the invention provides products of manufacture comprising or consisting of arrays or microarrays, and methods for making and using them. In alternative embodiments, the invention provides methods for making arrays or microarrays, and products of manufacture of the invention include arrays or microarrays made by methods of the invention.

In alternative embodiments, the invention provides products of manufacture comprising:

a solid, semi-solid, gel or gel-like, colloidal or sol-gel substrate substantially comprising a surface or a coat (or a coating) that prevents or inhibits cell attachment or is unable to sustain cell attachment, and the surface or coat (or coating) comprises a plurality of spots or circumscribed or defined areas capable of having a cell or cells attached thereto,

wherein optionally the surface or coat (or coating) preventing or inhibiting cell attachment comprises a polyacrylamide or a hydrogel, or a polyacrylamide-coated or hydrogel-coated surface, or a combination thereof,

and optionally the surface of the solid, semi-solid, gel or gel-like, colloidal or sol-gel substrate comprising a surface or a coat (or a coating) comprises or is a polyacrylamide-coated or hydrogel-coated glass or glass slide,

and optionally hydrogel-coated glass or glass slide are dehydrated before the spotting or printing,

and optionally the solid or semi-solid substrate comprises or consists of a glass or equivalent, and optionally the polyacrylamide or hydrogel are crosslinked onto a silane activated glass or glass slide;

and optionally the plurality of spots or circumscribed areas capable of having a cell or cells attach thereto comprises one or more compositions or mixtures of compositions capable of facilitating, initiating and/or sustaining attachment of the cells to the plurality of spots or circumscribed areas (and optionally the spot or circumscribed area comprising the one or more compositions capable of facilitating, initiating and/or sustaining attachment of the cells has the one or more compositions or mixtures of compositions directly attached to the solid, semi-solid, gel or gel-like, colloidal or sol-gel substrate, rather than on the surface or coat or coating preventing or inhibiting cell attachment),

and optionally the one or more compositions capable of facilitating, initiating and/or sustaining attachment of the cells comprises one or more extracellular-matrix proteins, or an extracellular-matrix-like composition or mixture of molecules, e.g., such as an interlocking mesh of fibrous proteins and glycosaminoglycans (GAGs), or proteoglycans (PG), heparan sulfate (HS), chondroitin sulfates (CS), keratan sulfate (KS), hyaluronic acid, collagen, elastin, fibronectin, laminin, a Cell Adhesion Molecule (CAM) or a CAM ligand, an integrin, a cadherin, a selectin, an addressin, or a mixture thereof, or equivalents thereof,

and optionally the cell is a mammalian cell or a human cell.

In alternative embodiments, the product of manufacture comprises or consists of, or is manufactured as, an array or microarray

In alternative embodiments, the invention provides products of manufacture made by a method comprising:

(a) providing a compound, a drug, a small molecule, or a small molecule drug, and a monomer solution capable of polymerizing;

(b) providing a solid, semi-solid, gel or gel-like, colloidal or sol-gel substrate;

(c) (i) mixing the compound, drug, small molecule or small molecule drug and the monomer solution, and optionally also including (or mixing in) a second solution or a composition capable of initiating and/or catalyzing polymerization of the monomer, and spotting or printing the mixture onto a plurality of circumscribed areas (such as spots) on the surface of the solid, semi-solid, gel or gel-like, colloidal or sol-gel substrate, or

(ii) spotting or printing the compound, drug, small molecule or small molecule drug onto a plurality of circumscribed areas (such as spots) on the surface of the solid, semi-solid, gel or gel-like, colloidal or sol-gel substrate, followed by spotting or printing a monomer solution capable of polymerizing over (on top of) substantially each spotted or printed compound, drug, small molecule or small molecule drug spot or print, wherein optionally:
(1) the monomer solution initially comprises a second solution or composition capable of initiating and/or catalyzing polymerization of the monomer, and the monomer solution polymerizes after the spotting or printing step,
(2) a second solution or composition capable of initiating and/or catalyzing polymerization of the monomer is spotted or printed over (on top of) substantially each spotted or printed compound, drug, small molecule or small molecule drug spot or print to initiate or catalyze polymerization of the monomer, or

(iii) spotting or printing the compound, drug, small molecule or small molecule drug onto a plurality of circumscribed areas (such as spots) on the surface of the solid, semi-solid, gel or gel-like, colloidal or sol-gel substrate, and the monomer solution is induced to polymerize by an external source, for example, by exposure to a light radiation, such as ultraviolet light, or heat;

wherein optionally the spotted or printed area for each compound, drug, small molecule or small molecule drug is approximately between about 0.01 mm² and 0.05 mm², or is approximately between about 0.02 mm² and 0.04 mm², or is approximately 0.03 mm²;

and optionally the spotting or printing of the mixture or monomer is done with an ink jetprinter or equivalent, or a liquid handling robot,

and optionally the compound, drug, small molecule or small molecule-mixture solution is prepared at a desired concentration and/or mixed with a pre-polymer solution or prepared in encapsulating lipids, liposomes or particles or nano-encapsulating particles,

and optionally the solution can be directly printed onto the dehydrated gel slides using a microarray robot; or the compound, drug, small molecule or small molecule drug/pre-polymer or the encapsulated compound, drug, small molecule or small molecule drug solution can be printed onto the dehydrated gel slides.

In alternative embodiments, the product of manufacture comprises or consists of, or is manufactured as, an array or microarray

In alternative embodiments, the invention provides products of manufacture product of manufacture comprising a combination of or a plurality of products of manufacture of the invention.

In alternative embodiments, a product of manufacture of the invention further comprises a substrate surface made by a method comprising spotting or printing one or more compositions capable of facilitating, initiating and/or sustaining attachment of the cells to the plurality of spots, prints or circumscribed areas, and optionally the one or more compositions capable of facilitating, initiating and/or sustaining attachment of the cells comprises one or more extracellular-matrix proteins, or an extracellular-matrix-like composition or mixture of molecules, e.g., such as an interlocking mesh of fibrous proteins and glycosaminoglycans (GAGs), or proteoglycans (PG), heparan sulfate (HS), chondroitin sulfates (CS), keratan sulfate (KS), hyaluronic acid, collagen, elastin, fibronectin, laminin, a Cell Adhesion Molecule (CAM) or a CAM ligand, an integrin, a cadherin, a selectin, an addressin, or a mixture thereof, or equivalents thereof, or a mixture thereof, or equivalents thereof.

In alternative embodiments, a product of manufacture of the invention further comprises placing or spotting or printing a cell or a plurality of cells to the plurality of spots, prints or circumscribed areas having layered thereon one or more compositions capable of facilitating, initiating and/or sustaining attachment of the cells, e.g., extracellular-matrix components, including proteins and/or polysaccharides, and/or an extracellular-matrix-like composition or mixture of molecules, e.g., such as an interlocking mesh of fibrous proteins and glycosaminoglycans (GAGs), or proteoglycans (PG), heparan sulfate (HS), chondroitin sulfates (CS), keratan sulfate (KS), hyaluronic acid, collagen, elastin, fibronectin, laminin, a Cell Adhesion Molecule (CAM) or a CAM ligand, an integrin, a cadherin, a selectin, an addressin, or a mixture thereof, or equivalents thereof, or a mixture thereof, or equivalents thereof.

In alternative embodiments, the invention methods for determining if a compound, drug, small molecule or small molecule drug has any or a desired effect or cellular response on a cell comprising:

(a) measuring or observing a cell (e.g., for any or a desired effect or cellular response on the cell) on a (“test”) product of manufacture (e.g., array, microarray) of any of claims 1 to 5 (wherein the product of manufacture comprises a compound, drug, small molecule or small molecule drug),

and optionally comparing the same cell on a “control” product of manufacture which is the same as a product of manufacture of any of claims 1 to 5 except that the “control” product of manufacture comprises: no compound, drug, small molecule or small molecule drug; a different compound, drug, small molecule or small molecule drug; a compound, drug, small molecule or small molecule drug known to have a different effect on the cell; or, the same compound, drug, small molecule or small molecule drug as on the “test” product of manufacture but at a different concentration;

(b) the method of (a), wherein the any or a desired effect or cellular response on a cell comprises cell death (e.g., apoptosis), secretion of bio-molecules (e.g., proteins, lipids, polysaccharides, nucleic acids and the like), proliferation (e.g., mitosis), cell-cell interaction, cell surface polypeptide turnover or recycling, vesicle release, membrane depolarization, ion (e.g., sodium, calcium or potassium) intracellular fluctuations or movement across cell membranes, and the like.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims.

FIG. 1 schematically illustrates an exemplary protocol for fabricating an exemplary small molecule microarray of the invention, as discussed in Example 1, below.

FIG. 2 schematically illustrates an exemplary protocol for fabricating an exemplary small molecule microarray of the invention, as discussed in Example 1, below.

FIG. 3 schematically illustrates an exemplary protocol for attaching small-molecule on to a product of manufacture of the invention, where the molecules are printed into a microarray represented on the first layer above the poly-acrylamide gel substrate, as discussed in Example 1, below.

FIG. 4 illustrates an exemplary composition of the invention (a product of manufacture), where the first two rows of the spots in A and B are immobilized fluorescent small-molecule spots, where the second rows are not immobilized; FIG. 4A illustrates the fluorescent small molecules pre-immerged in media solution, and FIG. 4B illustrates the fluorescent small molecules 10 minutes (min) post-immerged in media solution, as discussed in Example 1, below.

FIG. 5 illustrates an exemplary composition of the invention (a product of manufacture), where the fluorescent intensities of immobilized small-molecule microarray are imaged, FIG. 5A, at various time points while submerged in the media solution; the change of the fluorescent intensities are then plotted, as graphically illustrated in FIG. 5B, as discussed in Example 1, below.

FIG. 6 illustrates an exemplary composition of the invention (a product of manufacture), where a fluorescent anti-cancer drug doxorubicin, as illustrated in FIG. 6B, is printed at various concentrations in to an exemplary microarray, as illustrated FIG. 6A; as discussed in Example 1, below.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, the invention provides products of manufacture comprising or consisting of an array or microarray comprising a solid, semi-solid, gel or gel-like, colloidal or sol-gel substrate substantially comprising a surface, a coat or a coating that prevents cell attachment or are unable to sustain cell attachment, and the surface, coat or coating comprises a plurality of spots or circumscribed areas capable of having a cell or cells attach thereto.

In alternative embodiments, the invention provides products of manufacture comprising or consisting of an array or microarray comprising a solid, semi-solid, gel or gel-like, colloidal or sol-gel substrate substantially comprising a plurality of prints or spots, wherein substantially each print or spot comprises a multilayered “sandwiching” comprising: as a first layer on the solid, semi-solid, gel or gel-like, colloidal or sol-gel surface, which optionally can itself be (single or multi-) layered with a composition that inhibits or does not sustain or promote cell attachment (e.g., a polyacrylamide or equivalent), an immobilized small molecule (e.g., a drug) optionally embedded in a polymer (e.g., a polymerized monomer) (in one alternative embodiment, the circumscribed area or spot having the small molecule printed or spotted thereon does not have the composition that inhibits or does not sustain or promote cell attachment, e.g., a polyacrylamide or equivalent); and as a second layer a composition capable of promoting, initiating and/or sustaining cell attachment; and as a third layer a cell or a plurality of cells, wherein optionally the cell is a patient's cell, e.g., a normal or an abnormal cell, e.g., a cancer cell.

In alternative embodiments, the invention comprises a method for immobilizing small molecules (a plurality of small molecules) or other compounds in an array (e.g., a microarray) format for, e.g., screening, e.g., high throughput screening, of the effects of small molecule or other compounds and their combinations using a small number of cells. By manipulating the polymer(s) used for immobilization, the quantity and timing of the small molecule(s) released to the cells can be controlled. The controlled-release mechanism can be accomplished by changing the polymer properties, such as amount of polymerization and cross-linking chains, type of chains used, geometry of a mesh (or meshed array) created by the chains, chemical properties of the gel polymers, degradation of the polymer, and/or layers of polymers with different properties. In alternative embodiments, other methods to achieve the controlled-release mechanism comprise incorporation of immobilizing drug releasing nano-capsules or nano-particles, and/or micro-particles.

In alternative embodiments, individual compound(s) or compound mixtures that induce a cell response, e.g., a desired cellular response, can be identified. In alternative embodiments, induced cell responses comprise cell death (e.g., apoptosis), secretion of bio-molecules (e.g., proteins, lipids, polysaccharides, nucleic acids and the like), proliferation (e.g., mitosis), cell-cell interaction, cell surface polypeptide turnover or recycling, vesicle release, membrane depolarization and the like.

In alternative embodiments, uses (applications) for products of manufacture of the invention comprise:
Personalized therapy by screening the effects of drugs and their combinations on patients' normal, developing and/or diseased or infected cells.
Personalized screening for effects, e.g., desired effects or adverse effects, on patients' normal, developing and/or diseased or infected cells.
High throughput small molecule screening using small quantity of cells and compounds.

In alternative embodiments, uses of products of manufacture of the invention can significantly reduce the number cells and the amount of small molecule compounds needed in high throughput drug screening compared to conventional methods. For example, in one embodiment, the invention's array (e.g., microarray) format uses approximately 0.03 mm² culture area for each small molecule, less than 1/100 of the traditional unit culture area of approximately 3 mm², therefore reduce the number of cells required by over 100 times. As a result, this embodiment allows for the drug screening using limited cells from one patient, therefore providing a basis of the personalized therapy design. In one embodiment, the invention's array (e.g., microarray) format uses a much smaller amount of the compound screened, for example, one (1) nanoliter (nl) per test, or 1/1000 to the conventional volume of one (1) microliter (μl).

In alternative embodiments, products of manufacture of the invention comprise a controlled-release mechanism; for example, the quantity and timing of the compounds exposed to the cells can be controlled through the controlled-release mechanism. In alternative embodiments, this is done by changing the array (e.g., microarray) polymer properties such as amount of polymerization and crosslinking chains, type of the chains, geometry of the meshed created by the chains, chemical properties of the gel polymers, degradation of the polymer, layers of polymers with different properties, and the like.

In alternative embodiments, products of manufacture of the invention comprise a non-cell attaching substrate, extra-cellular matrix protein spots and “drug spots”; for example, the non-cell attaching substrate can provide a virtual barrier for cells. In one embodiment, cells are only present on the extra-cellular matrix protein spots. This can provide a well-defined reading area. The “drug spot” and the “extra-cellular matrix” spot may vary in size for various purposes.

In one embodiment, cells on an individual spot are well-isolated from cells on other spots; this prevents cells from migrating from one spot to another or inter-spot interactions and providing more dependable and independent results from each spot. This is a significant difference compared to the previous small-molecule microarray method.

### Robotic and Multi-Well Screening

### High Throughput Small Molecule Screening of the Invention

A substrate that prohibits cell attachment is chosen first. One embodiment comprises a polyacrylamide or hydrogel coated glass slide. The glass surface can be first activated using a silane. The polyacrylamide or hydrogel can be crosslinked onto the silane activated glass slides. The hydrogel coated slides can be dehydrated before the microarray printing. Thus, in one embodiment, products of manufacture of the invention comprise a polyacrylamide- or hydrogel-coated glass slide with a silane-activated surface.

Next, the compound or compound-mixture solution can be prepared at a desired concentration and/or mixed with a pre-polymer solution or prepared in nano-encapsulating particles. The solution can be directly printed onto the dehydrated gel slides using a microarray robot; or the compound/pre-polymer or the encapsulated compound solutions can be printed onto the dehydrated gel slides. Pure pre-polymer solutions can be printed on top of the compound/compound-mixtures and polymerization can be induced to immobilize the compounds. Various polymer chemistry or encapsulation can be designed to have the desirable drug holding and releasing parameters.

In alternative embodiments, products of manufacture of the invention comprise extra-cellular matrix proteins. The extra-cellular matrix proteins can be printed on top of the compound array, allowing the cells to attach.

In alternative embodiments, cells are seeded on the array, e.g., arrays having extra-cellular matrix proteins surfaces. In alternative embodiments, the cells on the array spots can attach to the extra-cellular matrix proteins surfaces.

Individual compound or compound mixtures that induce a desired cellular response can be identified by any means. Such responses may include but not limited to cell death (e.g., apoptosis), secretion of bio-molecules (e.g., proteins, lipids, polysaccharides, nucleic acids and the like), proliferation (e.g., mitosis), cell-cell interaction, cell surface polypeptide turnover or recycling, vesicle release, membrane depolarization, ion (e.g., sodium, calcium or potassium) intracellular fluctuations or movement across cell membranes, and the like.

In an alternative embodiment, the surface, coat or coating comprises a plurality of spots or circumscribed areas capable of having a cell or cells attach thereto, and these spots or circumscribed areas can be fabricated e.g., by substrate patterning, e.g., by optically creating an array of reactable spots. For example, in one embodiment, a glass plate is washed with an organosilane that absorbs to the glass to coat the glass. The organosilane coating is irradiated by deep UV light through an optical mask or by using a micromirror array that defines a pattern of an array. The irradiation cleaves the Si—C bond to form a reactive Si radical. Reaction with water causes the Si radicals to form polar silanol groups. The polar silanol groups constitute spots on the array and are further modified to couple other reactable molecules to the spots, as disclosed e.g., in U.S. Pat. Nos. (USPN) 5,324,591 and 6,653,124. For example, a silane containing a biologically 10 functional group such as a free amino moiety may be reacted with the silanol groups. The free amino groups are used as sites of covalent attachment for biomolecules such as extracellular-matrix proteins, or extracellular-matrix-like compositions or mixture of molecules.

In another embodiment, the surface, coat or coating comprises a plurality of spots or circumscribed areas capable of having a cell or cells attach thereto, and these spots or circumscribed areas can be fabricated e.g., by forming a substantially regular array of structures on a substrate as described in e.g., U.S. Pat. No. 6,649,491, where a surface layer of a first material is placed on a substrate of a second material, and the surface layer is sufficiently thin that stress fields at the interface of the surface layer and the substrate cause formation of separated regions; the at particle beam is directed on the surface layer and at a respective acute angle thereto to influence the direction of alignment of separated regions and/or the relative position of adjacent separated regions. By directing the particle beam on the surface layer the direction of alignment of separated regions and/or the relative position of adjacent separated regions is defined, and this provides the advantage that nanometer-scale structures having a high degree of regularity can be formed on the surface.

The invention will be further described with reference to the following examples; however, it is to be understood that the invention is not limited to such examples.

### EXAMPLES

### Example 1

### Making and Demonstrating Efficacy of Arrays of the Invention

The data presented herein demonstrates methods for making arrays, e.g., small-molecule microarrays, of this invention. In alternative embodiments, the invention provides small-molecule microarrays useful in drug screening in areas such as patient-specific drug screening and low-cost drug screening. The following describes the methods of creating such a small-molecule microarray and the resulting data.

In alternative embodiments, in order to screen the cellular response on the microarray, the cells should only be present on the microarray spot to have distinct read-outs. One method is to use a substrate of the microarray with a surface preventing the cell attachment. Poly-acrylamide coated glass slide is one of the options.

Because the cellular screening is conducted in solution, the small molecules need to be immobilized to prevent dissolving in the media solution. Exemplary methods are described herein.

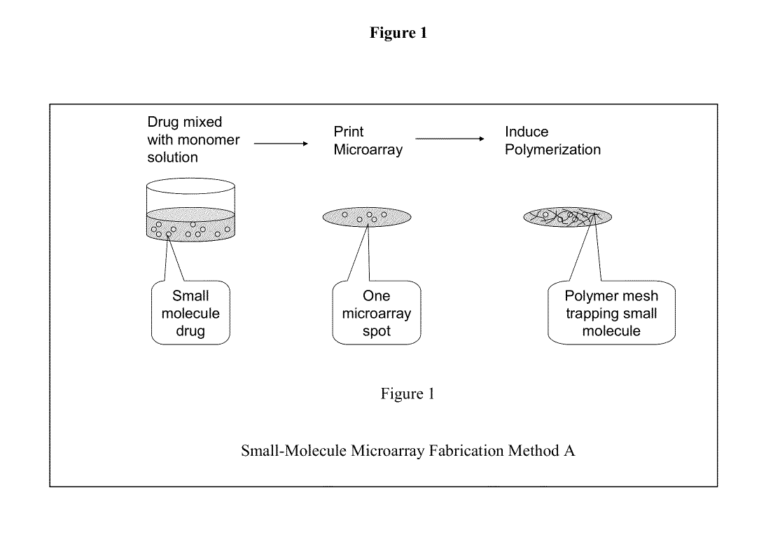

In the alternative embodiment illustrated in FIG. 1, the small molecule drug is mixed with a monomer solution; the drug-monomer mixture is then spotted onto a substrate in an array format; the polymerization process is then induced from the monomers, forming a polymer gel immobilizing the small molecule.

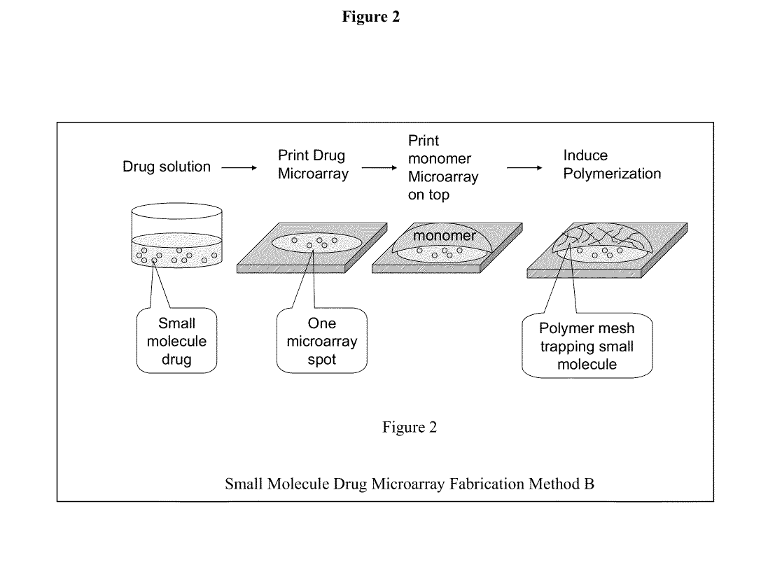

In the alternative embodiment illustrated FIG. 2, the small-molecule drug solution is first printed into microarrays on the substrate. The monomer solution is printed on top of each drug spot. Then the polymerization process is induced to cover and immobilizing the small-molecule drugs.

In one embodiment, after the small-molecule drugs are immobilized into a microarray format, a cell-attaching material is then printed on top of each spot. 25 Extracellular-matrix proteins, or an extracellular-matrix-like composition or mixture of molecules, e.g., such as an interlocking mesh of fibrous proteins and glycosaminoglycans (GAGs), or proteoglycans (PG), heparan sulfate (HS), chondroitin sulfates (CS), keratan sulfate (KS), hyaluronic acid, collagen, elastin, fibronectin, laminin, a Cell Adhesion Molecule (CAM) or a CAM ligand, an integrin, a cadherin, a selectin, an addressin, or a 30 mixture thereof, or equivalents thereof, or a mixture thereof, or equivalents thereof, are all alternative exemplary choices.

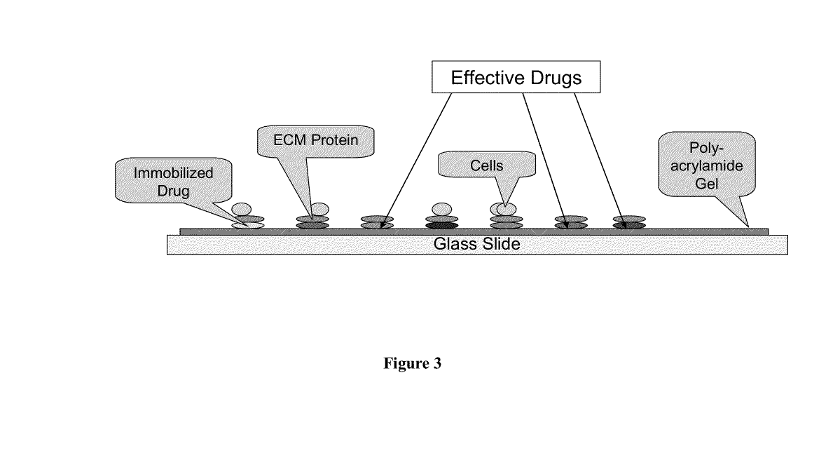

In the alternative embodiment illustrated in FIG. 3, the small-molecule drugs are printed into a microarray represented on the first layer above the poly-acrylamide gel substrate. The extracellular-matrix (ECM) proteins, or mix of proteins and GAGs, or proteoglycans (PG), heparan sulfate (HS), chondroitin sulfates (CS), keratan sulfate (KS), hyaluronic acid, collagen, elastin, fibronectin, 1 laminin, a Cell Adhesion Molecule (CAM) or a CAM ligand, an integrin, a cadherin, a selectin, an addressin, or a mixture thereof, or equivalents thereof, or a mixture thereof, or equivalents thereof, are spotted over each drug spot. The cells are then seeded on the microarray. Because the non-spotted poly-acrylamide surface does not allow cell attachment, the cells are only present on each spot.

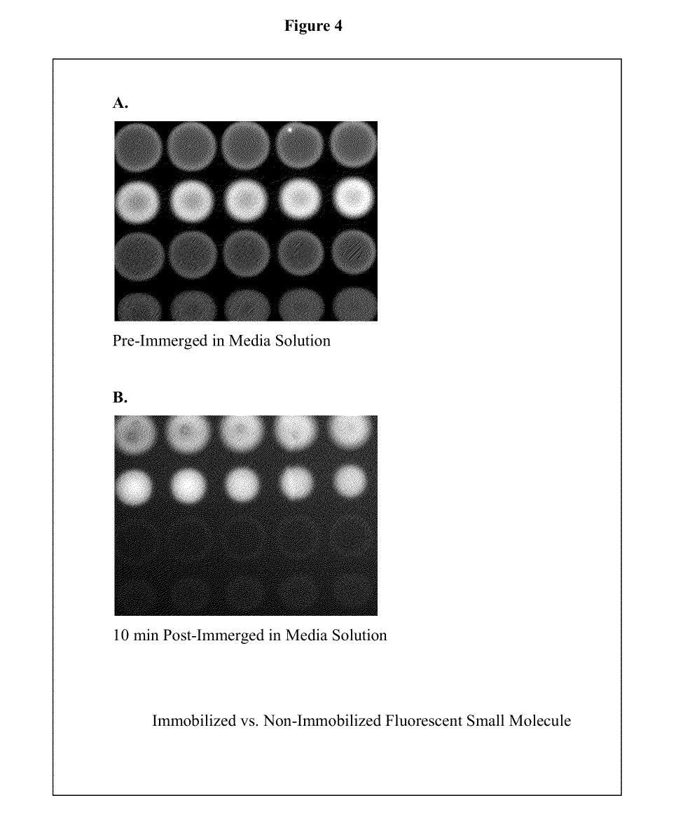

In the alternative embodiment illustrated in FIG. 4, the first two rows of the spots in A and B are immobilized fluorescent small-molecule spots, where the second rows are not immobilized. After submerged in the media solution for 10 min, the non-immobilized spots (second two rows) lost majority of their fluorescent signal, suggesting the majority of the molecules are removed from the spots. On the other hand, the immobilized the spots (first two rows) effectively retained their small-molecule contents represented by the sustained intensity of the fluorescent signals.

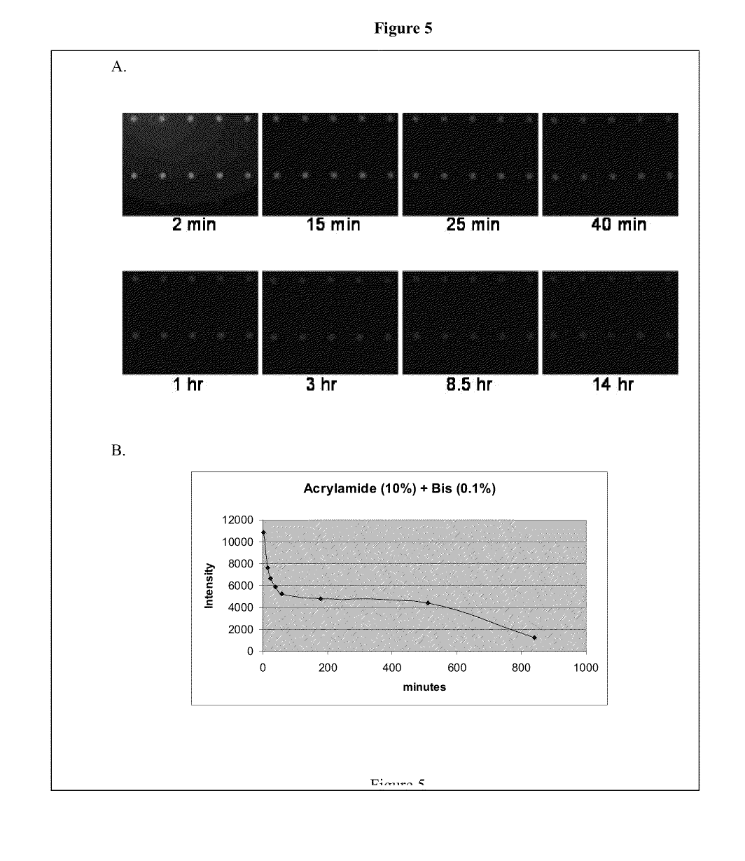

In the alternative embodiment illustrated in FIG. 5, the fluorescent intensities of immobilized small-molecule microarray are imaged (A) at various time points while submerged in the media solution. The change of the fluorescent intensities are then plotted (B). The loss of the fluorescent intensities represents the decreasing amounts of the small-molecules retained in the spots. The curve also suggests the amount of small-molecule released rate into the solution. This rate can be controlled by various factors such as the amount of polymerization and cross-linking, the degradation of the immobilizing polymer, the chemical properties and more.

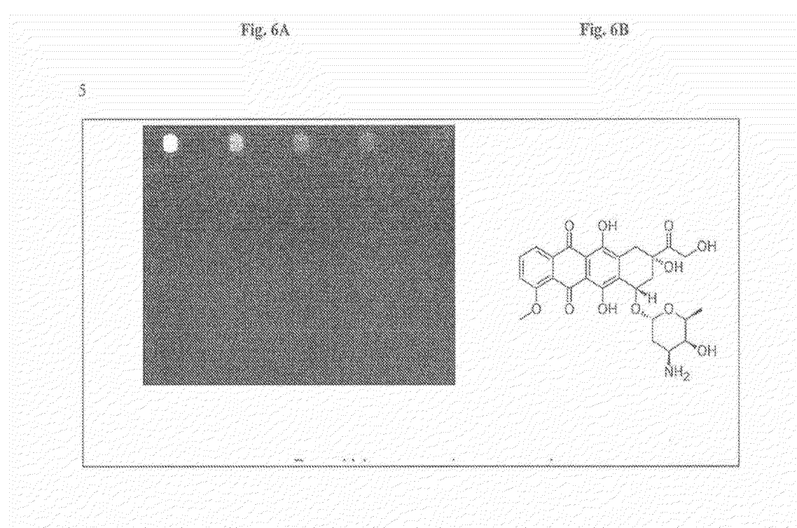

In the alternative embodiment illustrated in FIG. 6, a fluorescent anti-cancer drug doxorubicin is printed at various concentrations in to a microarray. The image was acquired after submerged in the media solution for 2 hours.

A number of embodiments of the invention have been described. Nevertheless, it will be understood that various modifications may be made without departing from the spirit and scope of the invention. Accordingly, other embodiments are within the scope of the following claims.

1. A product of manufacture comprising:
(a) a solid, semi-solid, gel or gel-like, colloidal or sol-gel substrate substantially comprising a surface, a coat or a coating that prevents or inhibits cell attachment or is unable to sustain cell attachment, and,
(b) the surface, coat or coating comprises a plurality of spots or circumscribed areas capable of having a cell or cells attached thereto,
and the plurality of spots or circumscribed areas capable of having a cell or cells attach thereto comprises one or more compositions or mixtures of compositions capable of facilitating, initiating and/or sustaining attachment of the cells to the plurality of spots or circumscribed areas,
and the spot or circumscribed area comprising the one or more compositions capable of facilitating, initiating and/or sustaining attachment of the cells comprises one or more compositions attached to the solid, semi-solid, gel or gel-like, colloidal or sol-gel substrate, rather than or not on the surface, coat or coating preventing or inhibiting cell attachment.

2. A product of manufacture made by a method comprising:
(a) providing a compound, a drug, a small molecule, or a small molecule drug, and a monomer solution capable of polymerizing;
(b) providing a solid, semi-solid, gel or gel-like, colloidal or sol-gel substrate; and
(c) (i) mixing the compound, drug, small molecule or small molecule drug and the monomer solution, and optionally also including or mixing in a second solution or a composition capable of initiating and/or catalyzing polymerization of the monomer, and spotting or printing the mixture onto a plurality of circumscribed areas or spots on the surface of the solid, semi-solid, gel or gel-like, colloidal or sol-gel substrate, or
(ii) spotting or printing the compound, drug, small molecule or small molecule drug onto a plurality of circumscribed areas or spots on the surface of the solid, semi-solid, gel or gel-like, colloidal or sol-gel substrate, followed by spotting or printing a monomer solution capable of polymerizing over or on top of substantially each spotted or printed compound, drug, small molecule or small molecule drug spot or print, wherein optionally:
(1) the monomer solution initially comprises a second solution or composition capable of initiating and/or catalyzing polymerization of the monomer, and the monomer solution polymerizes after the spotting or printing step,
(2) a second solution or composition capable of initiating and/or catalyzing polymerization of the monomer is spotted or printed over or on top of substantially each spotted or printed compound, drug, small molecule or small molecule drug spot or print to initiate or catalyze polymerization of the monomer, or
(iii) spotting or printing the compound, drug, small molecule or small molecule drug onto a plurality of circumscribed areas or spots on the surface of the solid, semi-solid, gel or gel-like, colloidal or sol-gel substrate, and the monomer solution is induced to polymerize by an external source, for example, by exposure to a light radiation, such as ultraviolet light, or heat.

3. A product of manufacture of claim 2, wherein one or more compositions capable of facilitating, initiating and/or sustaining attachment of the cells are spotted or printed with the compound, drug, small molecule or small molecule drug onto the plurality of circumscribed areas or spots on the surface of the solid, semi-solid, gel or gel-like, colloidal or sol-gel substrate,
and optionally the one or more compositions capable of facilitating, initiating and/or sustaining attachment of the cells comprise one or more extracellular-matrix proteins, or an extracellular-matrix-like composition or mixture of molecules,
and optionally the one or more compositions capable of facilitating, initiating and/or sustaining attachment of the cells comprises an interlocking mesh of fibrous proteins and glycosaminoglycans (GAGs), or proteoglycans (PG), heparan sulfate (HS), chondroitin sulfates (CS), keratan sulfate (KS), hyaluronic acid, collagen, elastin, fibronectin, laminin, a Cell Adhesion Molecule (CAM) or a CAM ligand, an integrin, a cadherin, a selectin, an addressin, or a mixture thereof, or equivalents thereof, or a mixture thereof, or equivalents thereof.

4. The product of manufacture of claim 2, further comprising a substrate surface made by a method comprising spotting or printing one or more compositions capable of facilitating, initiating and/or sustaining attachment of the cells to the plurality of spots, prints or circumscribed areas,
and optionally the one or more compositions capable of facilitating, initiating and/or sustaining attachment of the cells comprises one or more extracellular-matrix proteins, or an extracellular-matrix-like composition or mixture of molecules,
and optionally the one or more compositions capable of facilitating, initiating and/or sustaining attachment of the cells comprises an interlocking mesh of fibrous proteins and glycosaminoglycans (GAGs), or proteoglycans (PG), heparan sulfate (HS), chondroitin sulfates (CS), keratan sulfate (KS), hyaluronic acid, collagen, elastin, fibronectin, laminin, a Cell Adhesion Molecule (CAM) or a CAM ligand, an integrin, a cadherin, a selectin, an addressin, or a mixture thereof, or equivalents thereof, or a mixture thereof, or equivalents thereof.

5. The product of manufacture of claim 4, further comprising placing or spotting or printing a cell or a plurality of cells to the plurality of spots, prints or circumscribed areas having layered thereon one or more compositions capable of facilitating, initiating and/or sustaining attachment of the cells.

6. A method for determining if a compound, drug, small molecule or small molecule drug has any or a desired effect or cellular response on a cell comprising: measuring or observing a cell for any or a desired effect or cellular response on the cell after contact or exposure to a test product of manufacture, array, or microarray of claim 1,
wherein the product of manufacture comprises or has attached thereon the compound, drug, small molecule or small molecule drug.

7. The method of claim 6, further comprising comparing the same cell on a control product of manufacture, array, or microarray which is the same as the test product of manufacture, array, or microarray, except that the control product of manufacture, array, or microarray comprises:
no compound, drug, small molecule or small molecule drug; a different compound, drug, small molecule or small molecule drug; a compound, drug, small molecule or small molecule drug known to have a different effect on the cell; or,
the same compound, drug, small molecule or small molecule drug as on the test product of manufacture, array, or microarray but at a different concentration.

8. The method of claim 6, wherein the any or a desired effect or cellular response on a cell comprises: cell death; apoptosis; secretion of a bio-molecule, a protein, a lipid, a polysaccharide or a nucleic acid; proliferation; mitosis; a cell-cell interaction; a cell surface polypeptide turnover or recycling; a vesicle release; a membrane depolarization; or, an ion, a sodium, a calcium or a potassium intracellular fluctuations or movement across a cell membrane.

9. The method of claim 1, wherein the one or more compositions capable of facilitating, initiating and/or sustaining attachment of the cells comprises one or more of:
an extracellular-matrix protein or an extracellular-matrix-like composition or mixture of molecules; or,
an interlocking mesh of fibrous proteins and/or a glycosaminoglycan (GAG), a proteoglycan (PG), a heparan sulfate (HS), a chondroitin sulfate (CS), a keratan sulfate (KS), a hyaluronic acid, a collagen, an elastin, a fibronectin, a laminin, a Cell Adhesion Molecule (CAM) or a CAM ligand, an integrin, a cadherin, a selectin, an addressin, equivalents thereof, or a mixture thereof.

10. The method of claim 1, wherein the cell is a mammalian cell or a human cell.

11. The method of claim 1, wherein the product of manufacture comprises or consists of, or is manufactured as, an array or microarray.

12. The method of claim 1, wherein the surface, coat or coating preventing or inhibiting cell attachment comprises a polyacrylamide or a hydrogel, or a polyacrylamide-coated or hydrogel-coated surface.

13. The method of claim 1, wherein the surface of the solid, semi-solid, gel or gel-like, colloidal or sol-gel substrate comprising a surface, coat or coating is a polyacrylamide-coated or hydrogel-coated glass or glass slide.

14. The method of claim 13, wherein the hydrogel-coated glass or glass slide are dehydrated before the spotting or printing.

15. The method of claim 13, wherein the solid, semi-solid, gel or gel-like, colloidal or sol-gel substrate comprises or consists of a glass or equivalent, and optionally the polyacrylamide or hydrogel are crosslinked onto a silane activated glass or glass slide.

16. The method of claim 2, wherein the circumscribed, spotted or printed area for each compound, drug, small molecule or small molecule drug is approximately between about 0.01 mm² and 0.05 mm², or is approximately between about 0.02 mm² and 0.04 mm², or is approximately 0.03 mm².

17. The method of claim 2, wherein the spotting or printing of the mixture or monomer is done with an ink-jet printer or equivalent, or a liquid handling robot.

18. The method of claim 2, wherein the compound, drug, small molecule or small molecule-mixture solution is prepared at a desired concentration and/or mixed with a pre-polymer solution or prepared in encapsulating lipids, liposomes or particles or nano-encapsulating particles.

19. The method of claim 2, wherein the solution can be directly printed onto the dehydrated gel slides using a microarray robot; or the compound, drug, small molecule or small molecule drug/pre-polymer or the encapsulated compound, drug, small molecule or small molecule drug solution can be printed onto the dehydrated gel slides.

20. The method of claim 2, wherein the product of manufacture comprises or consists of, or is manufactured as, an array or microarray.
